(19) United States
(12) Patent Application Publication
(10) Pub. No.: US 2004/0030134 A1
(43) Pub. Date: Feb. 12, 2004

(54) Process for preparation of integrin receptor antagonist intermediates

(76) Inventors: James Christopher McWilliams, Basking Ridge, NJ (US); Elizabeth Buck, Somerville, NJ (US); Kan K. Eng, Yonkers, NY (US); Guy R. Humphrey, Hillsborough, NJ (US); Peter E. Maligres, Fanwood, NJ (US); Jess W. Sager, Bridgewater, NJ (US); Marjorie See Waters, Cranbury, NJ (US)

Correspondence Address: MERCK AND CO INC, P O BOX 2000, RAHWAY, NJ, 070650907

(21) Appl. No.: 10/398,364

(22) PCT Filed: Oct. 1, 2001
(86) PCT No.: PCT/US01/42411

(51) Int. Cl. C07D 471/02; C07D 487/02

(52) U.S. Cl. 544/218; 544/313; 544/314; 544/325; 546/113; 546/132

(57) ABSTRACT

A novel process is provided for the preparation of chiral intermediates useful in the asymmetric syntheses of αvβ3 integrin receptor antagonists. Also provided are the enantiomerically enriched intermediates that are obtained from the process.

### FIELD OF THE INVENTION

The present invention relates to a process for the efficient preparation of chiral allylic alcohol intermediates which are useful in the asymmetric synthesis of 9-substituted-3-(optionally substituted-aryl)-nonanoic acids. The process comprises an enantioselective 1,2-reduction of prochiral α,β-unsaturated ketones with a chiral reducing agent to afford chiral allylic alcohols which can be further processed into the desired substituted nonanoic acid derivatives, which are useful as αvβ3 integrin receptor antagonists for the inhibition of bone resorption and treatment and/or prevention of osteoporosis.

### BACKGROUND OF THE INVENTION

The present invention provides an efficient process for the preparation of chiral allylic alcohols of structural formula (1)

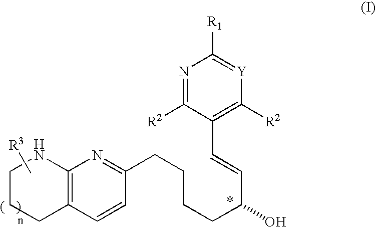

having the (R)-configuration at the stereogenic center marked with an *; wherein

n is 0, 1, or 2;

Y is CH or N;

R¹ is hydrogen, C₁₋₆ alkyl, or C₁₋₆ alkoxy;

R² is hydrogen, chloro, bromo, or iodo; and

R³ is selected from the group consisting of

hydrogen,

C₁₋₈ alkyl,

C₃₋₈ cycloalkyl,

C₃₋₈ cycloheteroalkyl,

C₃₋₈ cycloalkyl-C₁₋₆ alkyl, and

C₃₋₈ cycloheteroalkyl-C₁₋₆ alkyl.

The preparation of compounds of structural formula (I) in the racemic form was disclosed in U.S. Pat. No. 6,048,861 (Apr. 11, 2000), which is incorporated by reference herein in its entirety. The racemic allylic alcohols disclosed therein were converted in several steps into the desired 9-substituted-3-(optionally substituted-aryl)-nonanoic acids, which are useful as αvβ3 integrin receptor antagonists for the inhibition of bone resorption. The enantiomerically pure forms of the final products were obtained by means of HPLC resolution of the racemic mixture on a chiral solid support. Since only one antipode of the final product is preferred for use as an αvβ3 integrin receptor antagonist, the achiral process disclosed in U.S. Pat. No. 6,048,861 is inefficient in the sense that equal amounts of the less preferred enantiomer are obtained.

The present invention provides a process for the preparation of (R)-allylic alcohols of structural formula (1) in an efficient enantioselective fashion via 1,2-reduction of prochiral α,β-unsaturated ketones (enones) of structural formula (H) with a chiral reducing agent,

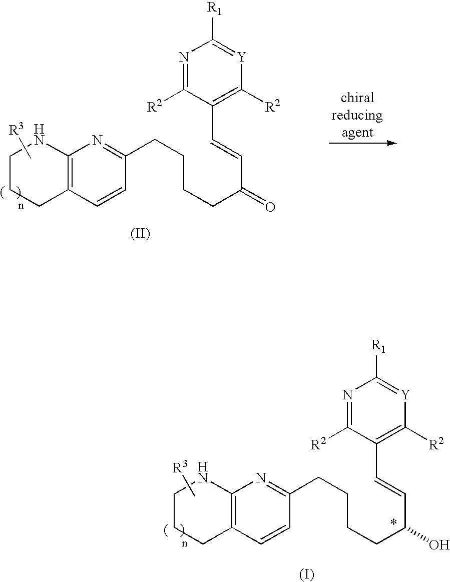

wherein n, Y, R¹, R², and R³ are as defined above.

### SUMMARY OF THE INVENTION

The present invention is concerned with a process for the preparation of chiral allylic alcohols of structural formula (I). The process utilizes an enantioselective chiral reducing agent under conditions that give rise to enhanced enantioselectivity in the reduction of prochiral α,β-unsaturated ketones (enones) of structural formula (II). The chiral allylic alcohols obtained in this fashion are key intermediates in the asymmetric synthesis of αvβ3 integrin receptor antagonists, which are useful for inhibiting bone resorption and treating and/or preventing osteoporosis.

### DETAILED DESCRIPTION OF THE INVENTION

The process of the present invention involves the preparation of chiral allylic alcohols of structural formula (I)

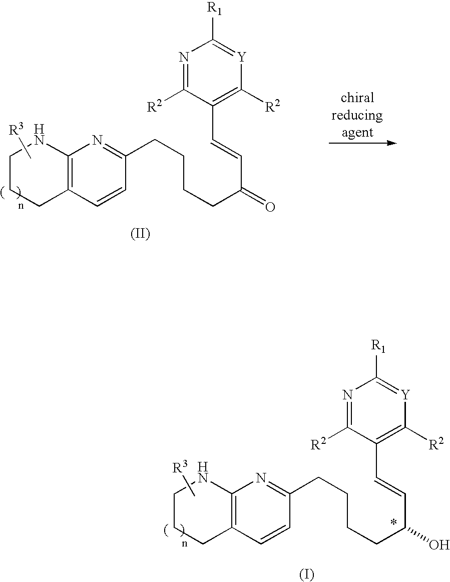

having the (R)-configuration at the newly formed stereogenic center marked with an

in an enantiomeric excess (ee) of at least 40% over the enantiomer having the opposite (S)-configuration, wherein

n is 0, 1, or 2;

Y is CH or N;

R¹ is hydrogen, C₁₋₆ alkyl, or C₁₋₆ alkoxy;

R² is hydrogen, chloro, bromo, or iodo; and

R³ is selected from the group consisting of

hydrogen,

C₁₋₈ alkyl,

C₃₋₈ cycloalkyl,

C₃₋₈ cycloheteroalkyl,

C₃₋₈ cycloalkyl-C₁₋₆ alkyl, and

C₃₋₈ cycloheteroalkyl-C₁₋₆ alkyl.

The process of the present invention comprises the step of treating an enone of structural formula (II) with an enantioselective chiral reducing agent in a reaction solvent in the presence of an organic polyamine, polyether, or polyaminoether modifier. The reaction solvent for the enone reduction is selected from the group consisting of diethyl ether, 1,4-dioxane, 1,2-dimethoxyethane (DME), methyl t-butyl ether (MTBE), diglyme, THF, toluene, dichloromethane, NMP, DMF, DMPU, and mixtures thereof. In one embodiment, the solvent for the enone reduction is THF; a mixture of THF and toluene; a mixture of THF, toluene, and dichloromethane; or a mixture of THF and dichloromethane.

In one embodiment of the process of the present invention, the enantioselective chiral reducing agent is a chiral aluminum hydride reagent prepared by mixing lithium aluminum hydride and approximately equimolar amounts of optically pure (R)-binaphthol and a proton source in an organic solvent. The organic solvent used in the preparation of the chiral aluminum hydride reagent can be diethyl ether, 1,4-dioxane, DME, MRBE, THF, toluene, or a mixture thereof.

The proton source is a compound of structural formula HXR⁴ wherein X is O, S, or NH and R⁴ is selected from the group consisting of

C₁₋₁₀ alkyl,

phenyl,

naphthyl,

pyridyl,

phenyl-C₁₋₃ alkyl,

phenyloxy-C₁₋₃ alkyl,

COR⁵,

SO₂R⁵,

P(O)R⁵(OR⁵), and

P(O)(OR⁵)2; and

each R⁵ is independently selected from the group consisting of

C₁₋₆ alkyl,

phenyl, and

phenyl-C₁₋₃ alkyl;

in which phenyl and alkyl are unsubstituted or substituted with one to three groups independently selected from C14 alkoxy, amino, and (C₁₋₄ alkyl)₁₋₂ amino.

In a class of this embodiment, the proton source HXR4 is a C₁₋₄ alkanol (X═O; R⁴ is C₁₋₄ alkyl). In a subclass of this class, the proton source is ethanol or methanol (X═O; R⁴ is Et or Me).

In a second embodiment of the process of the present invention, the chiral aluminum hydride reagent is a (R)-binaphthol-lithium aluminum hydride reagent of structural formula III:

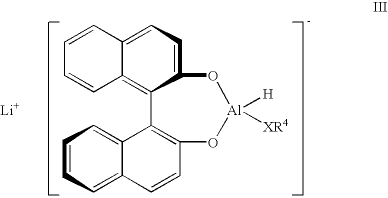

wherein

X is O, S, or NH;

R⁴ is selected from the group consisting of

C₁₋₁₀ alkyl,

phenyl,

naphthyl,

pyridyl,

phenyl-C₁₋₃ alkyl,

phenyloxy-C₁₋₃ alkyl,

COR⁵,

SO₂R⁵,

P(O)R⁵(OR⁵), and

P(O)(OR⁵)₂; and

each R⁵ is independently selected from the group consisting of

C₁₋₆ alkyl,

phenyl, and

phenyl-C₁₋₃ alkyl;

in which phenyl and alkyl are unsubstituted or substituted with one to three groups independently selected from C₁₋₄ alkoxy, amino, and (C₁₋₄ alkyl) 12 amino.

In one class of this embodiment, XR⁴ is OC₁₋₄ alkyl. In a subclass of this class, XR⁴ is OEt or OMe.

When XR⁴ represents an oxide residue, the chiral aluminum hydride reagents are known in the art as BINAL-H reagents [see R. Noyori et al., J. Am. Chem. Soc., 106: 6709-6716, 6717-6725 (1984); J. Am. Chem. Soc. 101: 3129-3131 (1979); and U.S. Pat. No. 4,284,581 (Aug. 19, 1981), the contents of each of which are incorporated by reference herein in their entirety]. The BINAL-H reagents developed by Noyori effect asymmetric reductions of prochiral ketones to chiral alcohols. Either the R- or S-antipode of the alcohol product is obtainable in a predictable fashion by choosing the proper handedness of the auxiliary binaphthol ligand. The BINAL-H reagent is prepared in situ from lithium aluminum hydride, optically pure 2,2′-dihydroxy-1,1′-binaphthyl, i.e., either (R)- or (S)-binaphthol (BIOL), and an alkanol. Preferred alkanols are methanol or ethanol. The addition of an organic polyamine, polyether, or polyaminoether modifier as disclosed in the present invention to the preformed chiral aluminum hydride reagent generates a “modified” BINAL-H reagent which exhibits unexpectedly improved enantioselectivities in the enone reduction over the “unmodified” BINAL-H reagent itself. Thus, while reductions of enones of structural formula (II) with an “unmodified” Noyori-type BINAL-H reagent proceed with enantioselectivities in the range of about 42-70% ee, the addition of an organic polyamine, polyether, or polyaminoether modifier to the BINAL-H reagent as in the present invention increases the enantioselectivity to a range of about 80-90%.

The “modified” BINAL-H reagent is prepared by mixing, for example, a lower alkanol, such as methanol or ethanol, and BINOL with a mixture of LAH in an organic solvent, such as toluene, THF, or a mixture thereof, which has been pretreated with THF. After heating the mixture for a specified period of time, the organic polyamine, polyether, or polyaminoether modifier is added and aged for a specified period of time.

In a class of this embodiment of the process of the present invention, the modified (R)-BINAL-H reagent is produced by adding ethanol or methanol (approximately one molar equivalent) and (R)-BINOL (slightly more than one molar equivalent) to a mixture of LAH in toluene or THF, which has been pre-treated with THF (>2 molar equivalents). After heating the mixture at 40-70° C. for about 30-90 minutes, an organic polyamine, polyether, or polyaminoether modifier is added and the mixture aged for about 30-90 minutes. The resultant mixture constitutes the modified enantioselective chiral reducing agent (“modified” R-BINAL-H), which is then reacted with a solution of the enone of structural formula (It). In a subclass of this class, the molar ratio of organic polyamine, polyether, or polyaminoether modifier to BINAL-H reagent is about 0.1:1 to about 3:1.

In a subclass of this class, the organic polyamine, polyether, or polyaminoether modifier is selected from the group consisting of 12-crown-4; bis-(2-dimethylaminoethyl)ether; triethylamine; (S)-(+)-1-(2-pyrrolidinyl)-pyrrolidine; 1,1,4,7,10,10-hexamethyltriethylenetetraamine (HMTTA); N,N,N′,N′-tetramethylethylenediamine (TMEDA); N,N,N′,N′-tetraethylethylenediamine (TEEDA); and N,N,N′,N′,N″-pentamethyldiethylenetriamine (PMDTA). Preferred organic polyamines are TMEDA and PMDTA.

In another class of this embodiment of the process of the present invention, the chiral reduction is carried out in a reaction solvent selected from the group consisting of diethyl ether, 1,4-dioxane, diglyme, DME, MTBE, THF, toluene, dichloromethane, DMF, NMP, DMPU, and mixtures thereof. In a subclass of this class, the reaction solvent for the reduction is THF; a mixture of THF and toluene; a mixture of THF, toluene, and dichloromethane; or a mixture of THF and dichloromethane.

In a further class of this embodiment, n is 1; Y is N; R² and R³ are hydrogen; R¹ is hydrogen or methyl; and XR⁴ is OEt or OMe.

In a further embodiment of the process of the present invention, the chiral reduction is carried out at a temperature in the range of about −100° C. to 40° C. In a class of this embodiment, the reaction is carried out at a temperature range of about −60° C. to 25° C.

In a further embodiment of the process of the present invention, the molar ratio of BINAL-H reagent to enone substrate is in the range of about 5:1 to about 1:1. In one class of this embodiment, the molar ratio of BINAL-H reagent to enone substrate is about 3:1.

Use of (S)-BINOL in place of (R)-BINOL generates the corresponding “modified” (S)-BINAL-H reagent. Treatment of the prochiral enone of structural formula (U) with a “modified” (S)-BINAL-H reagent yields the chiral allylic alcohol of structural formula (IV) having the (S)-configuration at the indicated newly formed stereogenic center,

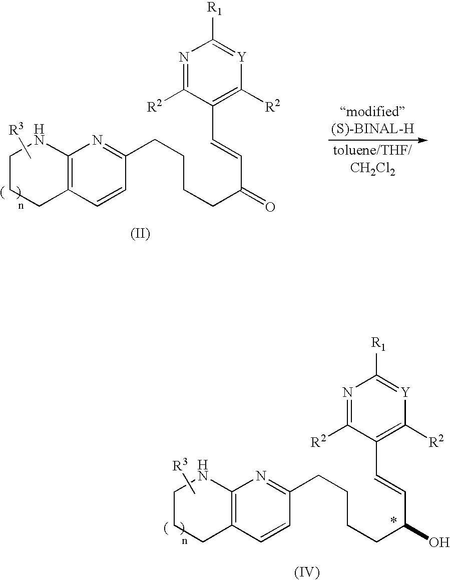

wherein n, Y, R¹, R², and R³ are as defined above.

A second aspect of the present invention provides for the preparation of chiral allylic alcohols of structural formula (I) by enantioselective reduction of enones of structural formula (II) wherein the chiral reducing agent is an (S)-oxazaborolidine of structural formula (V) in admixture with a source of borane,

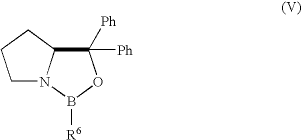

wherein R⁶ is hydrogen, C₁₋₄ alkyl, or C₁₋₄ alkoxy.

Chiral oxazaborolidines have been described in the art as enantioselective catalysts for the reduction of prochiral ketones [see E. J. Corey, et al., J. Amer. Chem. Soc., 109: 5551 (1987); E. J. Corey, et al., J. Amer. Chem. Soc., 109: 7925 (1987); D. J. Mathre, et al., J. Org. Chem., 58: 2880-2888 (1993) and references cited therein, which are incorporated by reference herein in their entirety].

In one embodiment of this second aspect of the present invention, R⁶ is hydrogen, methyl, or methoxy. Sources of borane include borane-dimethyl sulfide complex, catecholborane, dichloroborane-dimethyl sulfide complex, monochloroborane-dimethylsulfide complex, borane-THF complex, and 9-borabicyclo[3.3.1]nonane (9-BBN). The reaction is carried out in an organic solvent, such as dichloromethane, THF, toluene, or a mixture thereof, in the presence of an amine base, such as triethylamine and diisopropylethylamine, at a temperature in the range of about −60 to 25° C.

In a third aspect of the present invention, the compound of structural formula (I) is produced in an enantiomeric excess of about 80-90% over the enantiomer having the (S)-configuration at the stereogenic center. In one embodiment of this aspect of the present invention, the compound of structural formula (I) is produced in an enantiomeric excess of about 95% over the enantiomer having the (S)-configuration at the stereogenic center. The optical purity of the desired compound of structural formula (1) may be further increased by crystallization of residual amounts of the (R,S)-form from a suitable crystallization solvent system. In one embodiment, the crystallization solvent system is selected from the group consisting of acetonitrile; n-butyl acetate; ethyl acetate; isopropyl acetate; toluene; a mixture of ethyl acetate and acetonitrile; a mixture of ethyl acetate and heptane; a mixture of ethyl acetate and ethanol; a mixture of ethyl acetate and toluene; and a mixture of C₁₋₆ alkanol (which includes, but is not limited to, methanol, ethanol, n- and isopropanol, and n-, sec-, t-butanol) and toluene. In a class of this embodiment, the crystallization solvent system is a mixture of 2% to 5% n-propanol in toluene (v/v).

Yet another aspect of the process of the present invention comprises the following enantiomerically enriched compounds that are obtained from the process of the instant invention:

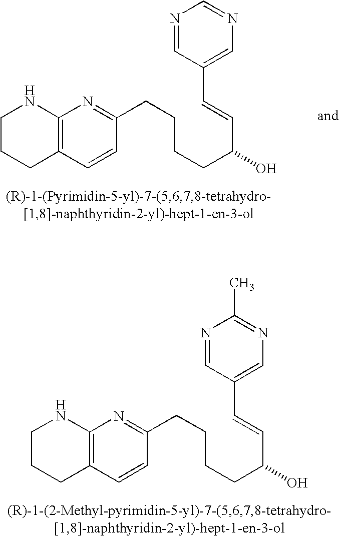

In one embodiment of this aspect of the present invention, there is provided the compound 1-(2-methyl-pyrimidin-5-yl)-7-(5,6,7,8-tetrahydro-[1,8]naphthyridin-2-yl)-hept-1-en-3-ol, comprising predominantly the (R)-enantiomer and a residual amount of the (S)-enantiomer, wherein the (R)-enantiomer is present in an enantiomeric excess of at least about 90% over the (S)-enantiomer. In a class of this embodiment, the (R)-enantiomer is present in an enantiomeric excess of at least about 98% over the (S)-enantiomer.

In another embodiment of this aspect of the present invention, there is provided the compound 1-(pyrimidin-5-yl)-7-(5,6,7,8-tetrahydro-[1,8]-naphthyridin-2-yl)-hept-1-en-3-ol, comprising predominantly the (R)-enantiomer and a residual amount of the (S)-enantiomer, wherein the (R)-enantiomer is present in an enantiomeric excess of at least about 90% over the (S)-enantiomer. In a class of this embodiment, (R)-enantiomer is present in an enantiomeric excess of at least about 98% over the (S)-enantiomer.

The chiral allylic alcohols of structural formula (I) of the present invention can be converted in the 3-step sequence of Claisen rearrangement, hydrogenation, and hydrolysis, as described in U.S. Pat. No. 6,048,861, into the final products of structural formula (VI), which are useful as αvβ3 integrin receptor antagonists,

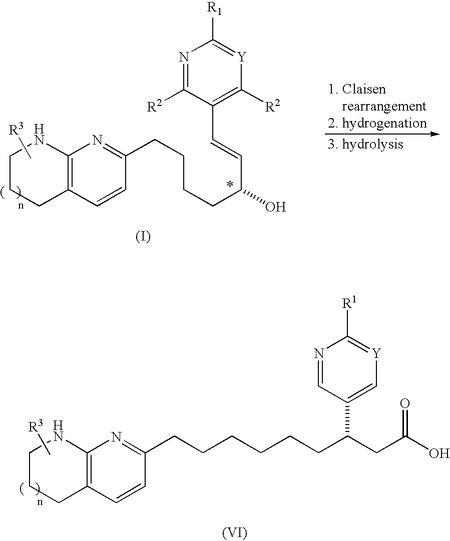

wherein n, Y, R¹, R², and R³ are as defined above.

The term “% enantiomeric excess” (abbreviated “ee”) shall mean the % major enantiomer less the % minor enantiomer. Thus, an 80% enantiomeric excess corresponds to formation of 90% of one enantiomer and 10% of the other. The term “enantiomeric excess” is synonymous with the term “optical purity.”

The term “enantioselective” shall mean a reaction in which one enantiomer is produced (or destroyed) more rapidly than the other, resulting in the predominance of the favored enantiomer in the mixture of products.

The term “alkyl” shall mean straight or branched chain alkanes of one to six total carbon atoms, or any number within this range (i.e., methyl, ethyl, 1-propyl, 2-propyl, n-butyl, sec-butyl, tert-butyl, etc.).

The term “alkoxy,” as used herein, refers to straight or branched-chain alkoxides of the number of carbon atoms specified (e.g., C₁₋₅ alkoxy) or any number within this range (i.e., methoxy, ethoxy, etc.).

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl, and cyclooctyl).

The term “cycloheteroalkyl” shall mean a 3- to 8-membered fully saturated heterocyclic ring containing one or two heteroatoms selected from N, O, and S. Examples of cycloheteroalkyl groups include, but are not limited to, piperidinyl, pyrrolidinyl, azetidinyl, morpholinyl, and piperazinyl.

Representative experimental procedures utilizing the novel process disclosed in the instant invention are detailed below. For purposes of illustration, the following examples are directed to the preparation of compounds 3 and 5, but doing so is not intended to limit the process of the present invention to the specific conditions for making these compounds. Proton and carbon-13 NMR spectra were recorded in CDCl₃ on a Bruker DPX 400. The chemical shifts are reported in ppm relative to residual CHCl₃ for proton (8=7.27) and CDCl₃ for carbon (δ=77.2). All coupling constants (J) are reported in Hertz (Hz) with proton multiplicities abbreviated as follows: s=singlet, d=doublet, t=triplet, m=multiplet, br=broad, o=overlapping. All temperatures are degrees Celsius unless otherwise noted.

Abbreviations: BINOL is 2,2′-dihydroxy-1,1′binaphthyl (1,1′-bi-2-naphthol); DBU is 1,8-diazabicyclo[5.4.0]undec-7-ene; DCM is dichloromethane (methylene chloride); DME is 1,2-dimethoxyethane; DMF is N,N-dimethylformamide; DMPU is 1,3-dimethyl-3,4,5,6-tetrahydro-2(1H)-pyrimidinone; ee is enantiomeric excess; HMTTA is 1,1,4,7,10,10-hexamethyltriethylenetetramine; HPLC is high-performance liquid chromatography; EPA is isopropanol; MTBE is methyl t-butyl ether; LAH is lithium aluminum hydride; NMP is 1-methyl-2-pyrrolidinone; NMR is nuclear magnetic resonance; PMDTA is N,N,N′,N′,N″-pentamethyldiethylenetriamine; TEEDA is N,N,N′,N′-tetraethylethylenediamine; THF is tetrahydrofuran; and TMEDA is N,N,N′,N′-tetramethylethylenediamine.

### EXAMPLE 1

Reduction of Enone (2) with “Modified” (R)-BINAL-H Reagent:

Step A: 1-(Pyrimidin-5-yl)-7-(5,6,7,8-tetrahydro-[1,8]-naphthyridin-2-yl)-hept-1-en-3-one (2)

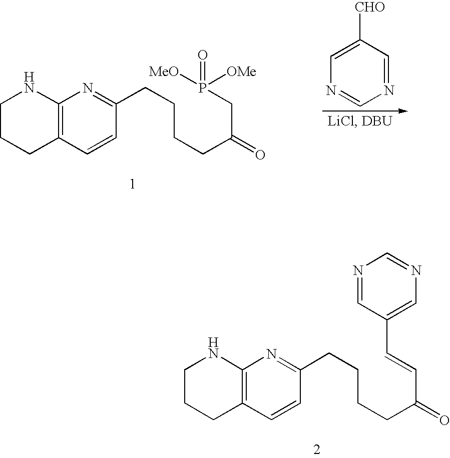

To a stirred suspension of anhydrous lithium chloride (3.54 g, 83.3 mmol) in acetonitrile (350 nmL) at room temperature was added a solution of ketophosphonate 1 (for preparation of 1, see U.S. Pat. No. 6,048,861) (28.3 g, 83.1 mmol) in acetonitrile (128 mL). After stirring for 15 min, a solution of DBU (9.52 mL, 64.1 mmol) in acetonitrile (32 mL) was added to produce a mostly fine white precipitate with some larger masses. The reaction mixture was briefly sonicated to break up the larger masses and stirred for 30 min. A solution of pyrimidine-5-carboxaldehyde (6.92 g, 64.1 mmol) in acetonitrile (128 mL) was added over 15 min. After 2 h, the reaction mixture was filtered and the filtrate concentrated. The residue was purified by flash chromatography (8% MeOH/EtOAc) to give 18.5 g (90%) of enone 2 as a yellow crystalline solid; m.p. 101-102° C.

¹H NMR (399.87 MHz, CDCl₃): δ 9.19 (s, 1H), 8.89 (s, 2H), 7.45 (d, J=16.3 Hz, 1H), 7.05 (d, J=7.3 Hz, 1H), 6.85 (d, J=16.3 Hz, 1H), 6.35 (d, J=7.3 Hz, 1H) 4.78 (br s, 1H), 3.39 (m, 2H), 2.72-2.67 (om, 4H), 2.58 (m, 2H), 1.89 (m, 2H), 1.79-1.72 (om, 4H) ppm.

¹³C NMR (100.55 MHz, CDCl₃): δ 199.3, 159.40, 159.36, 158.0, 155.9, 136.8, 134.7, 129.4, 128.8, 113.5, 111.5, 41.8, 41.6, 37.7, 29.5, 26.5, 23.9, 21.7 ppm.

Step B: Preparation of the “Modified” (R)-BINAL-H Reagent

To a dry 500 mL 3-neck round bottom flask at room temperature was added dry toluene (25 mL) followed by LAH (1.76 g, 46.4 mmol) under a nitrogen atmosphere. The resulting gray slurry mixture was treated with THF (7.2 mL), which was added over 10 min. at temperature <30° C. The resulting mixture was heated to 35° C. and treated with a solution of ethanol in toluene (6 M, 7.5 mL, prepared by adding 2.5 mL of ethanol in 4.9 mL of toluene), which was added slowly over 30 minutes between 35 and 40° C. After complete addition, the slurry was aged at 35° C. for 40 minutes and then cooled to 30° C. The resulting mixture was then treated with a solution of (R)-(+)-BINOL (12.3 g, 46 mmol) in toluene (90 ML) at 30° C., which was added at such a rate such that the batch temperature was maintained at <40° C., with cooling in an ice-bath if necessary. The resulting light gray slurry mixture was heated to 50° C. and aged for 1 hour and then allowed to cool to room temperature. The light gray mixture was then heated back up to 50° C. and treated with TMEDA (20.2 mL, 134 mmol) and stirred at 50° C. for 1 hour and then allowed to cool to room temperature. The total volume was 164 mL or ˜0.27 M solution of “modified” (R)-BINAL-H in toluene/THF solution. The solution was used directly in the following reduction step C without further purification.

Step C: (R)-1-(Pyrimidin-5-yl)-7-(5,6,7,8-tetrahydro-[1,8]-naphthyridin-2-yl)-hept-1-en-3-ol (3)

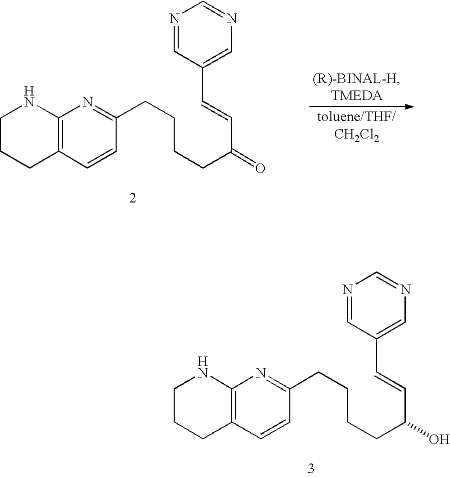

To a dry 500 mL 3-neck round bottom flask was added a toluene/THF solution of “modified” (R)-BINAL-H from Step B (0.27 M, 120 mL, 3.2 equiv.) under a nitrogen atmosphere, and the solution was cooled to −75 to −73° C. with a dry-ice acetone bath. Then a solution of enone 2 (3.3 g, 10.2 mmol) in DCM (23 mL) was added over 45 minutes while maintaining the batch temperature between −73 to −69° C. The reaction mixture was aged at −75° C. to −70° C. for 40 minutes and quenched with methanol (4 mL, 102 mmol) at −70° C. and then allowed to warn to room temperature. The reaction mixture was monitored by chiral HPLC: Chiralpak AD Analytical Column, 4.6×250 mm, 5 micron pore size; mobile phase: ethanol (with 0.1 v/v % diethylamine); flow rate: 2.0 mL/min.; injection volume=10 μL; detection=250 nm, sample preparation=100× dilution. Approximate retention times were:

| | |
|---|---|
| retention time (min.) | identity |

| | |
|---|---|
| 5.8 | (R)-allylic alcohol 3 |
| 6.9 | (S)-allylic alcohol 3 |
| 10.8 | enone 2 |

The reaction was deemed complete when the enone was <1.0 area %. The optical purity of (R)-3 was −80% enantiomeric excess (ee).

The reaction mixture was filtered through a pad of solka flock and the pad rinsed with DCM (20 mL). The resulting filtrate was transferred to a separatory funnel and extracted twice with aqueous tartaric acid solution (2.0 M, 1×100 mL and 1×50 mL). The combined aqueous phase was washed with DCM (20 mL). The pH of the washed aqueous phase was adjusted to 7 to 8 with 23 wt. % aqueous ammonium hydroxide solution and extracted with DCM (3×60 mL). The combined DCM solution was washed with 0.5 M ammonium chloride solution (3×100 mL) and dried over sodium sulfate. The solution was filtered and concentrated under reduced pressure to an oil. The resulting residue was dissolved in acetonitrile (100 mL) and concentrated to 10% of the initial volume and treated with additional acetonitrile (90 mL) and concentrated back to an oily residue.

The resulting residue (3.0 g) was charged into a 250-mL, 3 neck-round bottom flask, which was equipped with a temperature probe, a nitrogen inlet adapter, a magnetic stirrer, and a heating mantel, and treated with acetonitrile (60 mL) and then heated to 40° C. and aged 15 min. The resulting solution was then allowed to cool to room temperature and stirred overnight at room temperature.

The supernatant was checked by chiral HPLC assay at two wavelengths, 250 and 330 nm. After stirring at r.t. for 3 h, the (R)-allylic alcohol in acetonitrile solution was assayed to be 95% ee for (R)-3.

The slurry mixture was then cooled to 10° C. and filtered to isolate the (R)-allylic alcohol 3 as an acetonitrile solution (60 mL; 28 g/L; 1.7 g; 52% recovery) in a HPLC area % purity of 70% and in a chiral HPLC purity of 98% ee.

The HPLC purity (area%) was determined by gradient HPLC assay: YMCbasic AD Analytical Column, 4.6×250 mm, 5 micron pore size; Gradient Elution: Solvent A=5.0 mM each KH₂PO₄ and K₂HPO₄, Solvent B=Acetonitrile, T=0 min.@ 70% A:30% B. T=20 min.@ 20% A:80% B, T=21 min.@ 70% A:30% B; 1.0 mL/min.; injection volume=10 μL; detection=250 nm; sample preparation=100× dilution. Approximate retention times were:

| retention time (min.) | identity |
|---|---|
| 6.2 | (R)-allylic alcohol 3 |
| 7.9 | enone 2 |

An Alternative Procedure Using 2% n-Propanol in Toluene is Described Below:

The resulting residue after work-up of the reduction step (3.0 g) was charged into a 200-mL, 3 neck-round bottom flask, which was equipped with a temperature probe, a nitrogen inlet adapter, a magnetic stirrer, and a heating mantel, and treated with 2% n-propanol in toluene (36 mL) and heated to 45° C. and aged 15 min. The resulting solution was then allowed to cool to 34° C. and seeded with racemate (several crystals) and aged at 34° C. for 0.5 h and then cooled to room temperature and stirred overnight at room temperature.

The supernatant was checked by chiral HPLC assay at two wavelengths, 245 and 330 nm. After stirring at r.t. for overnight, the (R)-allylic alcohol in 2% n-propanol in toluene solution was assayed to be 98% ee for (R)-3.

The slurry mixture was then cooled to 17° C. and filtered to isolate the (R)-allylic alcohol 3 as a solution in 2% n-propanol in toluene (37 mL; 67 mg/mL; 2.48 g; 83% recovery) in a chiral HPLC purity of 98% ee.

Alcohol 3 could be obtained as a crystalline solid. This was accomplished by first solvent switching to toluene and then concentrating to a volume of 14 mL (5 volume per gram of 3) and treating the resulting solution with heptane solvent (19 mL), which was added dropwise over 0.5 h, to crystallize 3. Filtration of the resulting slurry mixture afforded 3 (2.36 g) in 78% isolated yield and 98% ee chiral purity.

¹H NMR (399.87 MHz, CDCl₃): δ 9.05 (s, 1H), 8.71 (s, 2H), 7.05 (d, J=7.3 Hz, 1H), 6.54 (d, J=16.1 Hz, 1H), 6.40 (dd, J=16.1, 5.5 Hz, 1H), 6.33 (d, J=7.3 Hz, 1H), 4.93 (br s, 1H), 4.38(m, 1H), 3.37 (m, 2H), 2.67 (t, J=6.3 Hz, 2H), 2.57 (t, J=7.4 Hz, 2H), 1.88 (m, 2H), 1.75-1.65 (om, 4H), 1.50 (m, 2H) ppm.

¹³C NMR (100.55 MHz, CDCl₃): δ 158.0, 157.4, 155.8, 154.5, 137.9, 137.0, 130.8, 122.4, 113.5, 111.5, 72.0, 41.8, 37.2, 37.0, 29.3, 26.5, 24.7, 21.6 ppm.

### EXAMPLE 2

Alternate Procedure for the Reduction Step C of Example 1

To a 0.731 molal slurry of unmodified BINAL-H slurry in THF/toluene (618 g, 452 mmol) was added 99% PMDTA (105 mL, 499 mmol) at 23° C. The resulting slurry was aged at 23° C. for 60 min. The slurry of modified BINAL-H was transferred to a slurry of 2 (44 g, 137 mmol) in THF (234 mL) over 30 min while cooling at −55° C. The slurry was allowed to warm to −37° C., whereupon acetic acid (31 mL) in n-heptane (173 mL) was added. The slurry was then warmed to 0° C., whereupon it was transferred to a flask containing 40 wt % citric acid (688 g). The mixture was aged overnight at 23° C. The aqueous phase was separated from the organic phase. The organic phase was extracted with 3.3 wt % citric acid (153 g). The combined aqueous phases were washed twice with toluene (1150 mL, 800 mL). The pH of the combined aqueous phases was adjusted to pH 7 with 50% NaOH (120 mL). The pH 7 aqueous phase was extracted twice with EtOAc (1 L, 400 mL), yielding a solution of allylic alcohols (38.5 g), with 3 present in 86% enantiomeric excess. Further enhancement of optical purity could be achieved as described in Example 1.

### EXAMPLE 3

Reduction of Enone (4) with “Modified” (R)-BINAL-H Reagent

Step A: Preparation of 2-methyl-pyrimidine-5-carboxaldehyde (9)

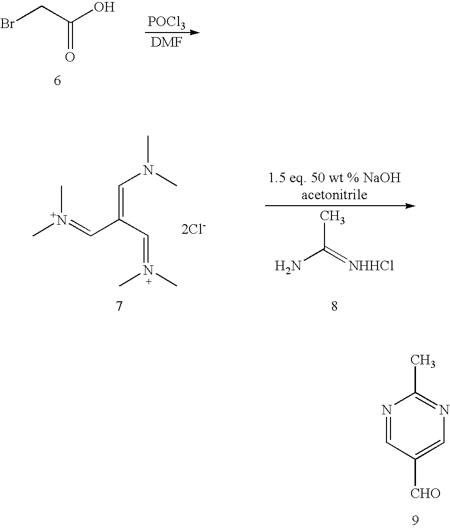

To a solution of bromoacetic acid 6 (12 g, 86.4 mmol) in DMF (44 mL) at 90° C. was added phosphorous oxychloride (24 mL, 260 mmol) over 5 h and then heated to 110° C. After stirring at 110° C. for 2.5 h, the mixture was cooled to 45° C. and quenched into a cold isopropanol (44 mL) at 2° C. and diluted with isopropyl acetate (44 mL) and then treated with water (6.2 mL), which was added over 45 minutes at 2° C. to form the dichloride vinamidinium salt 7. After stirring for 1 h, the deposited solid was collected and washed with isopropyl acetate (2×14 mL) and acetonitrile (2×14 mL) to afford 7 (12.0 g, 54%) as a pale yellow crystal.

To a slurry mixture of dichloride vinamidinium salt 7 (10.1 g, 39.9 mmole) and acetamidine hydrochloride 8.(4.2 g, 44.4 mmol) in acetonitrile (48 mL) at 22° C. was added 50% sodium hydroxide (4.9 g, 61.1 mmol) over 1.5 h and stirred at room temperature for 1.5 h.

The reaction mixture was monitored by HPLC: Zorbax® RX-C8 Analytical Column, 4.6×250 mm, 5 micron pore size, 60:40 Acetonitrile/5.0 mM each KH₂PO₄ and K₂HPO₄, 1.0 mL/min., injection volume=10 μL, detection=220 nm, sample preparation=100× dilution. Approximate retention times:

| retention time (min.) | identity |
|---|---|
| 2.95 | aldehyde 9 |
| 4.70 | acetamidine 8 |

The reaction mixture was filtered and washed with acetonitrile (10 mL), and the combined filtrate was concentrated under reduced pressure and solvent switched to heptane. The resulting heptane slurry mixture of crude 2 (25 mL) was extracted with methyl t-butyl ether (MTBE) (4×20 mL) at 40° C. The combined MTBE extract was filtered through a pad of fine silica gel and concentrated under reduced pressure. The residue was recrystallized from heptane to give aldehyde 2 (2.15 g, 44%) as pale yellow solid; m.p. 78-79° C.

¹H NMR (400.25 MHz, CDCl₃): δ 10.09 (s, 1H), 9.03 (s, 2H), 2.79 (s, 3H) ppm.

¹³C NMR (100.64 MHz, CDCl₃): δ 189.0, 173.2, 158.2, 126.3, 26.7 ppm.

Step B: 1-(2-Methylpyrimidin-5-yl)-7-(5,6,7,8-tetrahydro-[1,8]naphthyridin-2-yl)-hept-1-en-3-one (4)

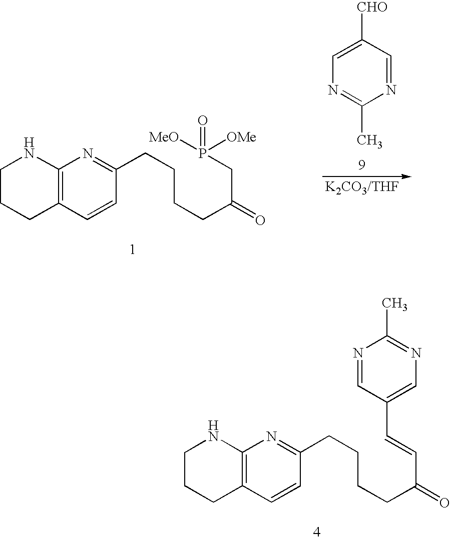

A stirred suspension of anhydrous powdered K₂CO₃ (6.21 g, 45 mmol), ketophosphonate 1 (for preparation of 1, see U.S. Pat. No. 6,048,861) (7.66 g, 22.5 mmol), and 2-methyl-pyrimidine-5-carboxaldehyde 9 (2.5 g, 20.5 mmol) in THF (250 mL) was heated at reflux for 4 h. After cooling to room temperature, the mixture was diluted with EtOAc (500 mL) and washed with water (100 mL) and brine (100 mL). The organic solution was dried over MgSO₄, filtered and concentrated. The residue was purified by flash chromatography (SiO₂; 10% EtOH/CH₂Cl₂) to give 5.66 g (85%) of the enone adduct 4 as a tan solid.

¹H NMR (400.13 MHz, CDCl₃): δ 8.77 (s, 2H), 7.42 (d, J=16.3 Hz, 1H), 7.04 (d, J=7.3 Hz, 1H), 6.80 (d, J=16.3 Hz, 1H), 6.34 (d, J=7.3 Hz, 1H) 4.80 (br s, 1H), 3.38 (m, 2H), 2.76 (s, 3H), 2.70-2.65 (om, 4H), 2.57 (m, 2H), 1.88 (m, 2H), 1.74-1.70 (om, 4H) ppm. ¹³C NMR (100.61 MHz, CDCl₃): δ 199.5, 169.4, 158.0, 156.0, 155.9, 136.8, 135.1, 128.4, 125.5, 113.4, 111.5, 41.8, 41.4, 37.7, 29.5, 26.5, 26.2, 24.0, 21.6 ppm.

Step C: (R)-1-(2-Methyl-pyrimidin-5-yl)-7-(5,6,7,8-tetrahydro-[1,8]-naphthyridin-2-yl)-hept-1-en-3-ol (5)

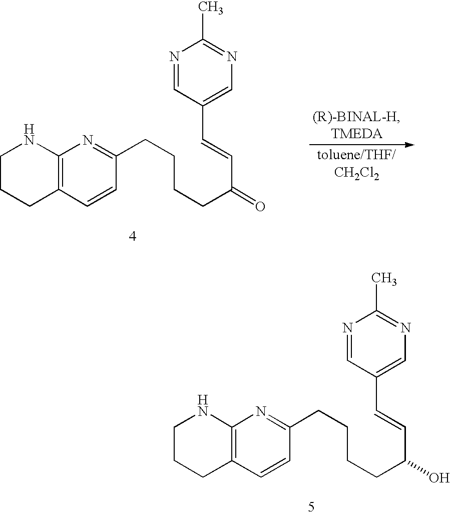

Toluene (390 mL) and THF (63 mL, 773 mmol) were charged to a flask containing lithium aluminum hydride (95 wt % purity, 15.4 g, 386 mmol). The slurry was stirred overnight. The slurry of lithium aluminum hydride was then gradually transferred to a slurry of (R)-(+)-1,1′-Bi-2-naphthol [(R)-Binol] (111 g, 388 mmol) in toluene (1.45 L). Ethanol (21 mL, 386 mmol) was then added to the mixture. The resultant slurry was stirred overnight. TMEDA (175 mL, 1160 mmol) was added to the slurry, and the mixture was stirred 60 min. The mixture was cooled in a −78° C. bath, whereupon enone 4 (60.4 g, 180 mmol) in dichloromethane (485 mL) was added. The reaction slurry was stirred 2 h while cooling in a −78° C. bath. The bath was then removed and the reaction was allowed to warm to ambient temperature, where it was stirred for an additional 30 min. The reaction was poured into a solution of Rochelle's salt (sodium potassium tartrate) (217 g) in water (2 L). The organic layer was separated and further washed twice with 2.5 N NaOH (313 mL/each) and once with water (100 mL). HPLC analysis of the organic layer indicated 87% of the allylic alcohols. Supercritical fluid chromatographic analysis indicated 5 was present in 82% enantiomeric excess.

### EXAMPLE 4

Alternate Procedure for the Reduction Step C of Example 3

To a flask containing 95% LiAlH₄ (3.84 g, 96.1 mmol) was added toluene (12 mL) and THF (27 mL). The slurry was heated to 55° C. and aged overnight. While continually heating at 50° C., EtOH (5.6 mL, 96 mmol) in THF (5.6 mL) was added over 60 min. After aging the slurry for an additional 60 min, a solution of (R)-Binol (28.3 g, 99 mmol) in THF (60 g) was added over 60 min. The slurry was aged for 60 min at 50° C., whereupon 99% PMDTA (22.3 mL, 107 mmol) was added all at once. The heating bath was removed and the slurry was allowed to age overnight. The slurry of modified BINAL-H reagent was transferred to a slurry of 4 (9.33 g, 27.7 mmol) in THF (49 mL) over 25 min while cooling at −60° C. The slurry was gradually warmed to −40° C. over 90 min and aged 70 min thereafter. Acetic acid (6.4 mL, 112 mmol) was added to the slurry. The slurry was warmed to −15° C., whereupon n-heptane (134 mL) was added, followed by 40 wt % citric acid. The mixture was allowed to warm to 20° C. and aged overnight. To the biphasic mixture was added DCM (50 mL). The aqueous phase was separated from the organic phase. The aqueous phase was washed with EtOAc (2×200 mL). The pH of the aqueous phase was adjusted to pH 7 with 50% NaOH. The aqueous was extracted with EtOAc (2×200 mL), providing a solution of allylic alcohols (82% assay yield), with 5 present in 90% enantiomeric excess.

TLC Rf=0.3 (80% CHCl₃/10% MeOH/10% EtOAc).

1H NMR (400 MHz, CDCl₃): δ 8.61 (s, 2H), 7.04 (d, J=7.3 Hz, 1H), 6.50 (d, J=16 Hz, 1H), 6.34 (dd, J=5.8, 16 Hz, 1H), 6.32 (d, J=7.3 Hz, 1H), 4.90 (br s, 1H), 4.37 (m, 1H), 3.64 (br s, 1H), 3.38 (m, 2H), 2.71 (s, 3H), 2.67 (t, J=6.3 Hz, 2H), 2.56 (t, J=7.4 Hz, 2H1), 1.88 (m, 2H), 1.66 (m, 4H), 1.48 (m, 2H) ppm.

¹³C NMR (100.55 MHz, CDCl₃): δ 166.7, 158.0, 155.7, 154.5, 136.8, 136.5, 127.5, 122.4, 113.3, 111.4, 72.0, 41.6, 37.2, 37.0, 29.3, 26.4, 25.7, 24.7, 21.5 ppm.

### EXAMPLE 5

Isolation of 99% ee (R)-1-(2-methyl-pyrimidin-5-yl)-7-(5,6,7,8-tetrahydro-[1,8]-naphthyridin-2-yl)-hept-1-en-3-ol (5) in Acetonitrile

A solution of 80% ee of 5 in methylene chloride (1.0 L; 22.8 g/L; 22.8 g, 70 mmol) was concentrated under reduced pressure to 100 mL and solvent switched to acetonitrile. The resulting slurry mixture was diluted with acetonitrile to a total volume of 750 mL (conc. ˜30 g/L) and heated to 65° C. and aged for 0.5 h or until all the solids dissolved into solution. The resulting solution was then allowed to cool to 45° C. and aged for 20 min. The resulting mixture was then allowed to cool to room temperature and stirred at room temperature for 2 h. The supernatant was checked by chiral HPLC: Chiralpak AD Analytical Column, 4.6×250 mm, 5 micron pore size; mobile phase: ethanol (with lv/v % diethylamine); flow rate: 2.0 mL/min.; injection volume=10 μL; detection=250 nm, sample preparation=100× dilution. Approximate retention times were:

| retention time (min.) | identity |
|---|---|
| 5.2 | (R)-allylic alcohol 5 |
| 7.4 | (S)-allylic alcohol 5 |

After stirring at room temperature for 2 hours, the (R)-allylic alcohol 5 in acetonitrile solution was assayed to be 99% ee. The slurry mixture was filtered to isolate the (R)-allylic alcohol 5 as an acetonitrile solution (745 mL; 22.9 g/L; 17.1 g; ˜75% recovery) in a HPLC area% purity of 89.3% and in a chiral purity of 99% ee.

¹H NMR (399.87 MHz, CDCl₃): δ 8.61 (s, 2H), 7.04 (d, J=7.3 Hz, 1H), 6.50 (d, J=16 Hz, 1H), 6.34 (dd, J=16.1, 5.8 Hz, 1H), 6.32 (d, J=7.3 Hz, 1H), 4.90 (br s, 1H), 4.37 (m, 1H), 3.64 (br s, 1H), 3.38 (m, 2H), 2.71 (s, 3H), 2.67 (t, J=6.3 Hz, 2H), 2.56 (t, J=7.4 Hz, 2H), 1.88 (m, 2H), 1.74-1.64 (om, 4H), 1.48 (m, 2H) ppm.

¹³C NMR (100.55 MHz, CDCl₃): δ 166.8, 158.1, 155.8, 154.6, 136.9, 136.6, 127.6, 122.5, 113.3, 111.5, 72.1, 41.7, 37.3, 37.1, 29.4, 26.5, 25.8, 24.8, 21.6 ppm.

### EXAMPLE 6

Isolation of 99% ee (R)-1-(2-methyl-parimidin-5-yl)-7-(5,6,7,8-tetrahydro-[1,8]-naphthyridin-2-yl)-hept-1-en-3-ol (5) in Wet Ethyl Acetate

A solution of 87% ee of 5 in methylene chloride (1.0 L; 22.8 g/L; 22.8 g, 67 mmol) was concentrated under reduced pressure to 268 mL and solvent switched to ethyl acetate. The total water content of the resulting ethyl acetate slurry mixture of 5 was adjusted to 1.5 v/v% by adding water (˜3.70 mL). The resulting slurry was stirred at room temperature for one to two hours.

The supernatant was checked by chiral HPLC assay at two wavelengths, 245 and 330 nm. After stirring at r.t. for one to two hours, the (R)-allylic alcohol in ethyl acetate with 1.5 v/v % water was assayed to be 99% ee for (R)5.

The slurry mixture was filtered to isolate the (R)-allylic alcohol 5 as a wet ethyl acetate solution (268 mL; 68 mg/mL; 18.2 g; 80% recovery) in a chiral HPLC purity of 99% ee.

Alcohol 5 could be obtained as a crystalline solid. This was accomplished by first solvent switching to heptane and then concentrating to a volume of ˜127 mL (7 volumes per gram of 5). Filtration of the resulting slurry mixture afforded 5 (17.3 g) in 76% isolated yield in a chiral purity of 99% ee.

### EXAMPLES 7-10

Other examples of “modified” BINAL-H reagents and their use in the chiral reductions of enone 4 are provided below:

3-Phenyl-1-propanol:

To a flask containing 95% LiAlH₄ (0.46 g, 11.5 mmol) was added THF (6 mL). The slurry was heated to 60° C., whereupon 98% 3-phenyl-1-propanol (1.6 mL, 11.6 mmol) was added over 60 min. After aging the slurry an additional 60 min, (R)-Binol (3.4 g, 11.9 mmol) in THF (7 g) was added over 60 min. The slurry was aged 60 rain at 60° C., whereupon TMEDA (3.5 mL, 23.2 mmol) was added, followed by toluene (10 mL). The heating bath was removed, and the slurry allowed to age overnight.

A solution of 4 (0.45 g, 1.34 mmol) in DCM (3.8 g) was added to the modified BINAL-H slurry at −65° C. After aging at −60° C. for 60 min, HPLC assay indicated 0.7 mmol of allylic alcohols, with 5 present in 87% enantiomeric excess.

2-Phenoxyethanol:

To a flask containing 95% LiAlH₄ (0.53 g, 13.3 mmol) was added THF (6 mL). The slurry was heated to 60° C., whereupon 98% 2-phenoxyethanol (1.7 mL, 13.3 mmol) was added over 60 min. After aging the slurry an additional 60 min, (R)-Binol (4.0 g, 13.9 mmol) in THF (8 g) was added over 60 min. The slurry was aged 60 min at 60° C., whereupon TMEDA (4.0 mL, 26.5 mmol) was added, followed by toluene (10 mL). The heating bath was removed, and the slurry allowed to age overnight.

A solution of 4 (0.72 g, 2.16 mmol) in DCM (6.1 g) was added to the modified BINAL-H slurry at −65° C. After aging at −60° C. for 60 min, HPLC assay indicated 0.5 mmol of allylic alcohols, with 5 present in 75% enantiomeric excess.

1-Decanol:

To a flask containing 95% LiAlH₄ (0.51 g, 12.8 mmol) was added THF (6 mL). The slurry was heated to 60° C., whereupon 99% 1-decanol (2.5 mL, 13.0 mmol) was added over 60 min. After aging the slurry an additional 60 min, (R)-Binol (3.8 g, 13.2 mmol) in THF (7.8 g) was added over 60 min. The slurry was aged 60 min at 60° C., whereupon TMEDA (3.9 mL, 25.9 mmol) was added, followed by toluene (10 mL). The heating bath was removed, and the slurry allowed to age overnight.

A solution of 4 (0.77 g, 2.30 mmol) in DCM (6.5 g) was added to the modified BINAL-H slurry at 65° C. After aging at −60° C. for 60 min, HPLC assay indicated 0.97 mmol of allylic alcohols, with 5 present in 84% enantiomeric excess.

1-Butanethiol:

To a flask containing 95% LiAlH₄ (0.50 g, 12.5 mmol) was added THF (6 mL). The slurry was heated to 60° C., whereupon 1-butanethiol (1.3 mL, 12.1 mmol) was added over 60 min. After aging the slurry an additional 60 min, (R)-Binol (3.7 g, 12.8 mmol) in THF (7.6 g) was added over 60 min. The slurry was aged 60 min at 60° C., whereupon TMEDA (3.8 mL, 25.2 mmol) was added, followed by toluene (10 mL). The heating bath was removed, and the slurry allowed to age overnight.

A solution of 4 (0.77 g, 2.30 mmol) in DCM (6.5 g) was added to the modified BINAL-H slurry at −65° C. After aging at −60° C. for 60 min, HPLC assay indicated 0.96 mmol of allylic alcohols, with 5 present in 41% enantiomeric excess.

### EXAMPLE 11

Reduction of Enone (4) with “Unmodified” (R)-BINAL-H Reagent:

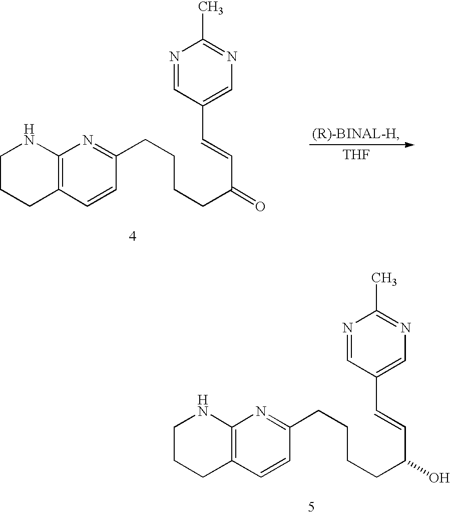

Lithium aluminum hydride (1 M solution in THF, 0.516 mL, 0.516 mmol) was slowly added to a solution of (R)-BINOL (0.15 g, 0.524 mmol) in THF (0.5 mL). Ethanol (0.03 mL, 0.521 mmol) was added to the resulting solution, and the mixture was aged for one hour at room temperature. Enone 4 (0.050 g, 0.16 mmol) was taken up in THF (1.2 mL) and cooled to −70° C. To this mixture was added the (R)-BINAL-H solution via cannula (exotherm to −63° C.). The reaction mixture was aged overnight at −70° C. and then quenched with 1 mL of methanol. Analysis of the crude reaction mixture indicated an 80% assay yield of the allylic alcohol, with the (R)-enantiomer present in 72% ee.

### EXAMPLE 12

Reduction of Enone (4) with (S)-Oxazaborolidine Reagent

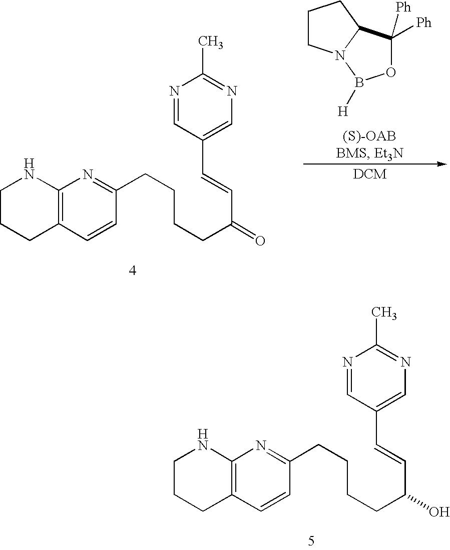

A 1.0 M solution of (S)-oxazaborolidine in toluene (1.5 mL, 1.5 mmol) and dichloromethane (6 mL) were charged to a flask under nitrogen. To this solution was added borane-methyl sulfide (0.14 mL, 1.5 mmol). An aliquot of this 0.2 M solution (4 mL, 0.8 mmol) was gradually added to a solution of enone 4 (202 mg, 0.60 mmol) and triethylamine (85 μL, 0.60 mmol) in dichloromethane (4 mL) while cooling at −40° C. After stirring 2 h, methanol (5 mL) was added, and the solution was gradually warmed to ambient temperature. Analysis of the crude reaction indicated a 42% assay yield of the allylic alcohol, with the (R)-enantiomer present in 92% enantiomeric excess.

What is claimed is:

1. A process for preparing a compound of structural formula (I) 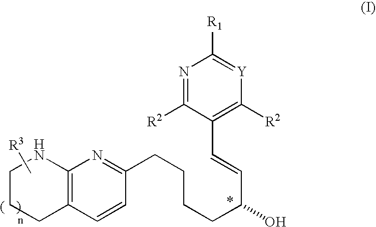
having the (R)-configuration at the stereogenic center marked with an *;
in an enantiomeric excess of at least 40% over the enantiomer having the (S)-configuration; wherein
n is 0, 1, or 2;
Y is CH or N;
R¹ is hydrogen, C₁₋₆ alkyl, or C₁₋₆ alkoxy;
R² is hydrogen, chloro, bromo, or iodo; and
R³ is selected from the group consisting of
hydrogen,
C₁₋₈ alkyl,
C₃₋₈ cycloalkyl,
C₃₋₈ cycloheteroalkyl,
C₃₋₈ cycloalkyl-C₁₋₆ alkyl, and
C₃₋₈ cycloheteroalkyl-C₁₋₆ alkyl;
comprising the step of treating a compound of structural formula (II) 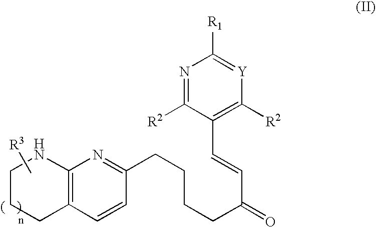
with an enantioselective chiral reducing agent, said process being carried out in a reaction solvent in the presence of an organic polyamine, polyether, or polyaminoether modifier at a temperature in the range of about −100° C. to 40° C.

2. The process of claim 1 wherein the enantioselective chiral reducing agent is a chiral aluminum hydride reagent prepared by mixing in an organic solvent lithium aluminum hydride and approximately equimolar amounts of (R)-binaphthol and a proton source of structural formula HXR⁴ wherein
X is O, S, or NH;
R⁴ is selected from the group consisting of
C₁₋₁₀ alkyl,
phenyl,
naphthyl,
pyridyl,
phenyl-C₁₋₃ alkyl,
phenoxy-C₁₋₃ alkyl,
COR⁵,
SO₂R⁵,
P(O)R⁵(OR⁵), and
P(O)(OR⁵)₂; and
each R⁵ is independently selected from the group consisting of
C₁₋₆ alkyl,
phenyl, and
phenyl-C₁₋₃ alkyl;
in which phenyl and alkyl are unsubstituted or substituted with one to three groups independently selected from C₁₋₄ alkoxy, amino, and (C₁₋₄ alkyl)₁₋₂ amino.

3. The process of claim 2 wherein said organic solvent is diethyl ether, MTBE, DME, diglyme, THF, toluene, or a mixture thereof.

4. The process of claim 2 wherein XR⁴ is OC₁₋₄ alkyl.

5. The process of claim 4 wherein XR⁴ is OEt or OMe.

6. The process of claim 2 wherein the chiral aluminum hydride reagent is a (R)-binaphthol-modified lithium aluminum hydride reagent of structural formula (III): 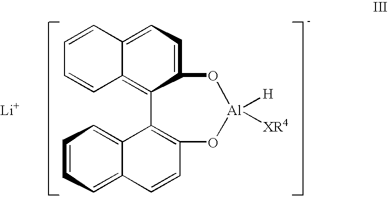
wherein
X is P, S, or NH;
R⁴ is selected from the group consisting of
C₁₋₁₀ alkyl,
phenyl,
naphthyl,
pyridyl,
phenyl-C₁₋₃ alkyl,
phenoxy-C₁₋₃ alkyl,
COR⁵,
SO₂R⁵,
P(O)R⁵(OR⁵), and
P(O)(OR⁵)₂; and
each R⁵ is independently selected from the group consisting of
C₁₋₆ alkyl,
phenyl, and
phenyl-C₁₋₃ alkyl;
in which phenyl and alkyl are unsubstituted or substituted with one to three groups independently selected from C₁₋₄ alkoxy, amino, and (C₁₋₄ alkyl) 12 amino.

7. The process of claim 6 wherein X is O.

8. The process of claim 7 wherein R⁴ is methyl or ethyl.

9. The process of claim 6 wherein the organic polyamine, polyether, or polyaminoether modifier is selected from the group consisting of 12-crown-4; bis-(2-dimethylamninoethyl)ether; triethylamine; (S)-(+)-1-(2-pyrrolidinyl)-pyrrolidine; 1,1,4,7,10,10-hexamethyltriethylenetetraamine; N,N,N′,N′-tetramethylethylenediamine (TMEDA); N,N,N′,N′-tetraethylethylenediamnine (TlEDA); and N,N,N′,N′,N″-pentamethyldiethylenetriamine (PMDTA).

10. The process of claim 9 wherein the organic polyamine modifier is TMEDA or PMDTA.

11. The process of claim 1 wherein said reaction solvent is selected from the group consisting of diethyl ether, 1,4-dioxane, MTBE, DME, diglyme, tetrahydrofuran, toluene, dichloromethane, DMF, DMPU, NMP, and mixtures thereof.

12. The process of claim 11 wherein said reaction solvent is THF; a mixture of THF and toluene; a mixture of THF, toluene, and dichloromethane; or a mixture of THF and dichloromethane.

13. The process of claim 1 wherein said temperature is in the range of about −60° C. to 25° C.

14. The process of claim 8 wherein n is 1; Y is N; R² and R³ are hydrogen; and R¹ is hydrogen or methyl.

15. The process of claim 14 wherein the organic polyamine modifier is TMEDA or PMDTA.

16. The process of claim 6 wherein the compound of structural formula (I) is produced in an enantiomeric excess of about 80-90% over the enantiomer having the (S)-configuration.

17. The process of claim 16 comprising the further step of removing residual amounts of the minor enantiomer having the (S)-configuration by crystallization of the (R,S)-form from a suitable crystallization solvent system.

18. The process of claim 17 wherein said crystallization solvent system is selected from the group consisting of acetonitrile; n-butyl acetate; ethyl acetate; isopropyl acetate; toluene; a mixture of ethyl acetate and acetonitrile; a mixture of ethyl acetate and heptane; a mixture of ethyl acetate and ethanol; a mixture of ethyl acetate and toluene; and a mixture of C₁₋₆ alkanol and toluene.

19. The process of claim 18 wherein said crystallization solvent system is a mixture of 2-5% n-propanol in toluene.

20. The compound 1-(2-methyl-pyrimidin-5-yl)-7-(5,6,7,8-tetrahydro-[1,8]-naphthyridin-2-yl)-hept-1-en-3-ol, comprising predominantly the (R)-enantiomer and a residual amount of the (S)-enantiomer, wherein the (R)-enantiomer is present in an enantiomeric excess of at least about 90% over the (S)-enantiomer.

21. The compound of claim 20 wherein the (R)-enantiomer is present in an enantiomeric excess of at least about 98% over the (S)-enantiomer.

22. The compound 1-(pyrimidin-5-yl)-7-(5,6,7,8-tetrahydro-[1,8]naphthyridin-2-yl)-hept-1-en-3-ol, comprising predominantly the (R)-enantiomer and a residual amount of the (S)-enantiomer, wherein the (R)-enantiomer is present in an enantiomeric excess of at least about 90% over the (S)-enantiomer.

23. The compound of claim 22 wherein the (R)-enantiomer is present in an enantiomeric excess of at least about 98% over the (S)-enantiomer.

24. The process of claim 2 wherein the molar ratio of said chiral aluminum hydride reagent to said compound of structural formula (I) is about 3:1.

25. The process of claim 2 wherein the molar ratio of said organic polyamine, polyether, or polyaminoether modifier to said chiral reducing agent is about 0.1:1 to about 3:1.
